(19) United States
(12) Patent Application Publication
(10) Pub. No.: US 2009/0234181 A1
(43) Pub. Date: Sep. 17, 2009

(54) Assessment Tool For Storing and Sharing Music Therapy Patient Records

(75) Inventor: Mijin Kim, Hartsdale, NY (US)

Correspondence Address: DUANE MORRIS LLP - NY;PATENT DEPARTMENT, 1540 BROADWAY, NEW YORK, NY, 10036-4086, US

(73) Assignee: Institute For Music and Neurologic Function

(21) Appl. No.: 12/404,099

(22) Filed: Mar. 13, 2009

Related U.S. Application Data
(60) Provisional application No. 61/036,645, filed on Mar. 14, 2008.

(51) Int. Cl. A61M 21/00

(52) U.S. Cl. 600/28

(57) ABSTRACT

This invention relates to an assessment tool for use in musical therapy comprising: (a) at least one patient record stored within the assessment tool; (b) at least one field or datum within the patient record; (c) a security scheme that either permits or denies authorization a user to view and/or edit each field or datum within each patient record; and optionally, (d) a plurality of users of the assessment tool each with a unique identification. This invention also relates to combining the above embodiments with a computer to store the patient records, and the security scheme. This above tool may also be connected to a network, including the internet, via the computer element.

This application claims benefit of U.S. Provisional Ser. No. 61/036,645 filed Mar. 14, 2008, the entire disclosure of which is hereby incorporated by reference.

### BACKGROUND OF THE INVENTION

Music therapy is a health care profession that serves a variety of clinical populations, including patients with psychological, psychiatric, neurological, developmental, rehabilitative and other medical conditions. In the U.S., music therapists are licensed and board-certified. Music therapists have standardized training and qualifications. However, music therapists lack a standardized assessment tool to measure the progress of their patients. The lack of such a tool hampers their ability to gather clinical progress data easily and employ it for outcome-oriented research. To make such a tool available especially in an online computerized form would make it even more useful for patient monitoring and research purposes.

There is a need for a novel assessment tool for music therapists who work with patients suffering from neurological and psychological disabilities. The development of such a tool could help to advance the delivery of music therapy services as part of comprehensive care in the U.S. and around the world, particularly in the area of clinical care of physically and psychologically disabled people of all ages. Such a tool will advance the use and effectiveness of music therapy and thereby improve the function of people with neurological disorders such as stroke, Parkinson's disease, and dementia.

### SUMMARY OF THE INVENTION

The invention described herein is an assessment tool for use in musical therapy comprising

(a) at least one patient record stored within the assessment tool;

(b) at least one field or datum within the patient record;

(c) a security scheme that either permits or denies authorization a user to view and/or edit each field or datum within each patient record; and optionally,

(d) a plurality of users of the assessment tool each with a unique identification.

Another embodiment of the invention combines either of the above embodiments with a computer to store the patient records, and the security scheme.

Another embodiment further limits the above embodiment wherein the computer is connected to a network comprising at least one other computer, including the internet.

### DETAILED DESCRIPTION OF THE INVENTION

Here we present the invention of a novel assessment tool for music therapists who work with patients suffering from neurological and psychological disabilities. The assessment tool described herein allows music therapists to measure the progress of their patients. The assessment tool also allows for consistent record keeping of initial patient assessments and ongoing treatment evaluations of music therapy patients. Additionally, the assessment tool serves as a repository for information and data collected during the course of treatment for an individual patient or for a group of patients. Furthermore, the assessment tool also allows music therapists to share data collected in the evaluation and treatment of their patients with other therapists and researchers.

The tool allows the therapist to share access to patients' records with nearly any person while maintaining a desired level of patient privacy. Such privacy is tailored to the recipient of patient's records. The access to patient records is based on the authority level of the user and his relationship to the particular patient. The tool permits access based on the authority level of the user of the tool, providing particular types of information to a given user while also protecting particular types of data and information from that user. The tool also only permits authorized users to enter and edit data. The tool can share patient information with therapists, administrators and researchers yet maintain patient privacy, especially as required by the Health Insurance Portability and Accountability Act of 1996.

### Patient Data

Music therapists use a variety of methods to observe and evaluate their patients. Such methods include but are not limited to clinic evaluations using scales, recorded informal clinic observations, recorded case studies prepared by therapists, audio recordings of sounds, speech and music, recordings of video, and photographs.

Any known scale of clinical evaluation can be used with the assessment tool. It is also expected that future scales can be adapted for use with the invention. Non-limiting examples of such scales that can be used with the invention include the Western Aphasia Battery and the Test of Adolescent/Adult Word Finding (both available from Pro-Ed, Austin Tex.).

The Western Aphasia Battery is a scale that identifies and measures the severity of aphasia syndromes in adolescents and adults. The scale evaluates a patient's responses for information content, fluency, auditory comprehension, repetition, and naming. The Western Aphasia Battery yields an Aphasia Quotient that can be used to categorize patients according to various aphasia taxonomic classifications and determine the degree of severity. Known characteristics of behavior and recovery can be associated with each classification.

The Test of Adolescent/Adult Word Finding is an individually administered scale that assesses an important expressive vocabulary skill: word finding. Therapists use this test to diagnose word-finding disorders quickly and reliably. The test has five core sections: Picture Naming: Nouns; Picture Naming: Verbs; Sentence Completion Naming; Description Naming; and Category Naming. Optionally, the test can be used with a special sixth comprehension section that allows the examiner to determine if errors are a result of word-finding problems or are due to poor comprehension. The test provides formal and informal analyses of two dimensions of word finding: speed and accuracy. The formal analysis yields standard scores, percentile ranks, and grade standards for item response time. The informal analysis yields secondary characteristics (gestures and extra verbalization) and substitution types. Speed can be measured in actual or estimated item response time.

Informal analysis such as described above can be recorded with the assessment tool. Such informal analysis may be guided by a scale or kit such as is possible with the Test of Adolescent/Adult Word Finding. However, informal analysis can also be performed and recorded based on a therapist's own expert skills and knowledge. Such informal analysis can be recorded in written, audio or video formats or combinations thereof.

The assessment tool can store case studies conducted by therapists. A case study is generally an in-depth, longitudinal examination of a single patient. Such a case study provides a systematic way of observing a patient, collecting data and analyzing the information. A case study may optionally present, as well as test, a hypothesis. Such case studies can be recorded in written, audio or video formats, or combinations thereof.

Audio recordings can be stored by the assessment tool. Such audio recordings can be of but are not limited to music, singing, speech, sounds or combinations thereof. Non-limiting subjects of audio recordings include patients, therapists, assistants to the therapist, musicians, instruments, animals, nature, machines, phenomena or any combination thereof. The subject can be a single person or thing or a group of persons or things.

Video recordings can be stored by the assessment tool. Such video recordings can be of, but are not limited to therapy sessions, performances, practice sessions, common situations, experiments, and concerts. The video recordings can be real-time motion, slow motion, time-lapse, still images or combinations thereof. Video recordings can be photographs. The video recordings can be of any part or the entire electromagnetic spectrum, including but not limited to the full visual spectrum of light, black and white only, grayscale, infrared, ultraviolet, single hues or combinations thereof. Non-limiting subjects of video recordings include the patient, the therapist, assistants to the therapist, musicians, instruments, animals, nature, machines, phenomena or any combination thereof. The subject can be a single person or thing or a group of persons or things.

### Patient Fields

In addition to data, patient records also include fields that describe non-clinical information about the patient. An individual datum while stored in the assessment tool can be paired with a particular patient and other fields. Such fields include, but are not limited to patient name, patient identification code, patient date of birth, patient gender, patient age, patient address, physical characteristics of the patient, information about a patient's family, diagnosis therapist, assisting therapist, primary physician, social worker, date of music therapy initial assessment, date of treatment session, music therapy diagnosis (e.g., treatment areas focused), type of music therapy (e.g., focused treatment group vs. individual, length of sessions), treatment application code (e.g., therapeutic techniques utilized), frequency of assessment and evaluation, site of treatments, people present at session, case number, medical evaluations, neurological evaluations, family history, case progress, treatment costs and combinations thereof.

The assessment tool can generate reports based on a search for any of the fields or data stored. Such reports can include but are not limited to patient history, outcome based statistical studies, case studies, demographic and attendance patterns, cost-effectiveness, effectiveness of specific music therapy techniques, neurological investigations, and refining of therapeutic strategies.

### Security

The invention allows therapists to share patient records with others and dictate a desired level of privacy for various categories of patient information. Each user of the assessment tool is assigned a unique user identification. Each user identification is paired with a scheme of authorization that is divided by patient and field/datum. A given user can be blocked from reading or editing access to any field or datum for a particular patient. A given user can also be blocked from reading or editing a particular type of field or datum for all patients. A given user can also be blocked from reading or editing a particular field or datum for a particular patient.

When a user requests to see a given field or datum within a given patient's record, the assessment tool verifies that the user has authority to view that particular field or datum within that particular patient's record. If the user is deemed to be authorized to view the particular field or datum of the particular patient the information is presented to the user. If the user is not deemed to be authorized to view the particular field or datum of the particular patient, a message stating that the information is privileged is presented.

When a user returns to the tool a given field or datum within a given patient's record as changed from its previous state, the assessment tool verifies that the user has editing authorization for that particular field or datum within that particular patient's record. If the user is deemed to be authorized to edit the particular field or datum of the particular patient the field or datum is updated within the assessment tool. If the user is not deemed to be authorized to edit the particular record of the particular patient, a message stating that editing is not permitted is displayed to the user, and no change is recorded in the assessment tool.

When a user returns to the tool a given field or datum within a given patient's record as a new field or a previously null field now containing data, the assessment tool verifies that the user has editing authorization for that particular field or datum within that particular patient's record. If the user is deemed to be authorized to edit or add the particular field or datum of the particular patient the field or datum is updated within the assessment tool. If the user is not deemed to be authorized to edit or add the particular record of the particular patient, a message stating that editing is not permitted is displayed to the user, and no change is recorded in the assessment tool.

According to U.S. Federal regulations enacted under the Health Insurance Portability and Accountability Act of 1996, health care providers may not use or disclose protected health information except as specifically permitted in or required by 45 C.F.R. § 164.502, hereby incorporated by reference in its entirety. Furthermore, barring specifically excepted situations, U.S. Federal regulations require that when using or disclosing protected health information, providers must make reasonable efforts to limit protected health information to the minimum required to accomplish the intended purpose. Certain embodiments of the present invention use the security feature of the assessment tool to prevent data fields containing protected health information from being viewed by users not authorized to view such information. These embodiments of the present invention allow at least one user to specify which data fields are accessible to other specific users or groups of users. The security system can be implemented so that only the minimum required protected health information is shared between authorized users.

### DEFINITIONS

The term “user” as used herein refers to any person who interacts with the assessment tool to read a field or datum, enter a field or datum, delete a field or datum, write a field or datum, create a field or datum, add a field or datum, edit a field or datum, and/or copy a field or datum of any patient record.

The term “patient record” as used herein refers to a collection of information having at least one field or datum.

The term “field” as used herein refers to information stored in the assessment tool and related to any aspect of a patient, including but not limited to patient name, patient identification code, patient date of birth, patient gender, patient age, patient address, physical characteristics of the patient, information about a patient's family, diagnosis therapist, assisting therapist, primary physician, social worker, date of music therapy initial assessment, date of treatment session, music therapy diagnosis, type of music therapy, treatment application code, frequency of assessment and evaluation, site of treatments, people present at session, case number, medical evaluations, neurological evaluations, family history, case progress, treatment costs and combinations thereof.

What is claimed:

1. An assessment tool for use in musical therapy comprising:
(a) at least one patient record stored within the assessment tool;
(b) at least one field or datum within the patient record; and
(c) a security scheme that either permits or denies authorization a user to view and/or edit each field or datum within each patient record.

2. The assessment tool of claim 1, further comprising a plurality of users of the assessment tool each with a unique identification.

3. The assessment tool of claim 2, wherein a computer is used to store at least one patient record.

4. The assessment tool of claim 2, wherein a computer is used to implement the security scheme.

5. The assessment tool of claim 4, wherein the security scheme either permits or denies authorization to view each field or datum within each patient record for each user.

6. The assessment tool of claim 4, wherein the security scheme either permits or denies authorization to edit each field or datum within each patient record for each user.

7. The assessment tool of claim 3, wherein the computer is connected to a network comprising at least one other computer.

8. The assessment tool of claim 4, wherein the computer is connected to a network comprising at least one other computer.

9. The assessment tool of claim 7, wherein the network is the internet.

10. The assessment tool of claim 8, wherein the network is the internet.

11. The assessment tool of claim 3, wherein at least one field or datum stored comprises an audio recording.

12. The assessment tool of claim 3, wherein at least one field or datum stored comprises a video recording.

13. The assessment tool of claim 2, wherein at least one field or datum stored is an evaluation of a patient on a scale of clinical evaluation.

14. The assessment tool of claim 13, wherein the scale of clinical evaluation is the Western Aphasia Battery.

15. The assessment tool of claim 13, wherein the scale of clinical evaluation is the Test of Adolescent/Adult Word Finding.
